(19) United States
(12) Patent Application Publication
(10) Pub. No.: US 2026/0102064 A1
(43) Pub. Date: Apr. 16, 2026

(54) OPHTHOSCOPE

(71) Applicant: Dean McGee Eye Institute, Oklahoma City, OK (US)

(72) Inventors: Jibran Sharieff, Oklahoma City, OK (US); Brandon Kingrey, Edmond, OK (US)

(21) Appl. No.: 19/359,382

(22) Filed: Oct. 15, 2025

Related U.S. Application Data
(60) Provisional application No. 63/707,627, filed on Oct. 15, 2024.

(51) Int. Cl. A61B 3/12; A61B 3/00; A61B 3/14

(52) U.S. Cl. CPC A61B 3/1208; A61B 3/0008; A61B 3/14

(57) ABSTRACT

A novel mobile device attachment that allows the mobile device to obtain photography and videography of the fundus oculi. This mobile device attachment does not require condensing lenses or an additional power source as it redirects the native mobile device light source using a series of three mirrors to be in line with the camera of the mobile device. Ultimately, this novel mobile device attachment creates an upright, non-inverted, magnified image of the fundus oculi.

### CROSS REFERENCE TO RELATED APPLICATION

This application claims priority from and the benefit of U.S. Provisional Application 63/707,627 filed Oct. 15, 2024, which is incorporated herein by reference.

### BACKGROUND

The ophthalmoscope is an instrument used to examine the fundus oculi of the eye. The fundus oculi, hereinafter may be referred to interchangeably with the term “fundus,” includes the retina, optic disc, and blood vessels in the internal posterior portion of the eye. Examination of the fundus oculi can help to diagnose various eye conditions and systemic conditions, including but not limited to glaucoma, macular degeneration, retinal artery occlusions, retinal vein occlusions, optic neuritis, hypertensive retinopathy, diabetic retinopathy, and elevated intracranial hypertension.

The direct ophthalmoscope is a device that allows for a magnified and narrow field of view of the fundus oculi that is right side up and not inverted. The indirect ophthalmoscope allows for binocular viewing of the fundus oculi with a wider field of view of the fundus oculi that is upside down and inverted. The indirect ophthalmoscope requires a convex lens to obtain this wider field of view. The direct ophthalmoscope often does not require pupillary dilation, but the indirect ophthalmoscope often does require pupillary dilation. Camera technology has allowed digital images of the fundus oculi using principles similar to the optics used in direct and indirect ophthalmoscopes. Many of these fundus cameras are large instruments that are not portable, but there are a few portable fundus cameras available. Some of these portable fundus cameras have incorporated attachments to a smartphone's camera to obtain an image of the fundus with a smartphone. The majority of these smartphone fundus cameras are similar to the indirect ophthalmoscope because they require condensing lenses to obtain a view of the fundus.

### SUMMARY

The optical accessory device and method of this disclosure pertain to a smartphone attachment that allows for photography and videography of the fundus oculi. The optical accessory device functions similarly to a direct ophthalmoscope because it produces a magnified, upright, and non-inverted image of the fundus without using any condensing lenses. Other smartphone fundus camera attachments incorporate one or multiple lenses to obtain a view of the fundus.

These other smartphone fundus camera attachments also require a power source, such as batteries, to power a separate from the smartphone, which is needed to obtain images of the fundus. The optical accessory design of this disclosure utilizes the built-in smartphone camera flash to obtain fundus images and videos rather than a light source associated with the optical accessory or separate from both the smartphone and optical accessory. This is achieved by redirecting the smartphone flashlight to be nearly coaxial with the camera of the smartphone. Many modern smartphones now have magnets for accessories on the back of the smartphone. The device of this disclosure can attach to these smartphones through this magnetic attachment. Advantageously, the optical accessory device of this disclosure produces a magnified, upright, non-inverted fundus image, works with an un-dilated pupil, and does not require condensing lenses, batteries, or additional power sources.

### BRIEF DESCRIPTION OF THE DRAWINGS

FIG. 1 is a rear-facing view of the ophthalmoscope optical accessory according to some embodiments of this disclosure.

FIG. 2 is a front-facing view of the optical accessory of FIG. 1.

FIG. 3 is a front-facing, isometric view of the optical accessory of FIG. 1 with the housing split to illustrate a face plate and a base plate.

FIG. 4 is a rear-facing isometric view of the optical accessory of FIG. 1 with the housing split to illustrate the face plate and the base plate.

FIG. 5 is a rear-facing view of the optical accessory and a case.

FIG. 6 is a front-facing view of the optical accessory and the case.

FIG. 7 is a front-facing, isometric view of the optical accessory and the case.

FIG. 8 is a rear-facing, isometric view of the optical accessory and the case.

FIGS. 9, 10 and 11 are images of an optical accessory and an associated case.

### DETAILED DESCRIPTION

The drawings included with this application illustrate certain aspects of the embodiments described herein. However, the drawings should not be viewed as exclusive embodiments. The subject matter disclosed is capable of considerable modifications, alterations, combinations, and equivalents in form and function, as will occur to those skilled in the art with the benefit of this disclosure.

The present disclosure may be understood more readily by reference to these detailed descriptions. For simplicity and clarity of illustration, where appropriate, reference numerals may be repeated among the different figures to indicate corresponding or analogous elements. In addition, numerous specific details are set forth in order to provide a thorough understanding of the various embodiments described herein. However, it will be understood by those of ordinary skill in the art that the embodiments described herein can be practiced without these specific details. In other instances, methods, procedures, and components have not been described in detail so as not to obscure the related relevant feature being described. Also, the description is not to be considered as limiting the scope of the embodiments described herein. The drawings are not necessarily to scale and the proportions of certain parts have been exaggerated to better illustrate details and features of the present disclosure.

Before discussing the presently disclosed inventive concepts in detail by way of exemplary description, drawings, experimentation, and results, it is to be understood that the inventive concepts disclosed herein are not limited in application to the details of construction and the arrangement of the compositions, formulations, steps, or components set forth in the following description or illustrated in the drawings, examples, experiments, and/or results. The presently disclosed inventive concepts are capable of other embodiments or of being practiced or carried out in various ways. As such, the language used herein is intended to be given the broadest possible scope and meaning; and the embodiments are meant to be exemplary, not exhaustive. Also, it is to be understood that the phraseology and terminology employed herein is for the purpose of description and should not be regarded as limiting except where indicated as such.

All of the compositions, devices, systems, and/or methods disclosed herein can be made and executed without undue experimentation in light of the present disclosure. Although certain steps are described herein and illustrated in the figures as occurring sequentially, some steps may occur simultaneously with each other or in an order that is not depicted. While the compositions, devices, systems, and/or methods of the presently disclosed inventive concepts have been described in terms of particular examples and embodiments, it will be apparent to those of skill in the art that variations may be applied to the compositions, devices, systems, and/or methods and in the steps or in the sequence of steps of the methods described herein without departing from the concept, spirit, and scope of the presently disclosed inventive concepts. All such similar substitutes and modifications apparent to those skilled in the art are deemed to be within the spirit, scope, and concept of the presently disclosed inventive concepts as defined by, but not limited to, the appended claims.

As utilized in accordance with the present disclosure, the following terms, unless otherwise indicated, shall be understood to have the following meanings: the use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” As used in this specification and claims, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements, or method steps.

Throughout this disclosure, the terms “about”, “approximate”, and variations thereof, are used to indicate that a value includes the inherent variation or error for the device, system, the method being employed to determine the value, or the variation that exists among the study subjects. For example, but not by way of limitation, when the terms “about” or “approximately” are utilized, the designated value may vary by +/−twelve percent, or +/−eleven percent, or +/−ten percent, or +/−nine percent, or +/−eight percent, or +/−seven percent, or +/−six percent, or +/−five percent, or +/−four percent, or +/−three percent, or +/−two percent, or +/−one percent, or +/−one-half percent. As used herein the symbol “+/−” indicates “plus or minus”.

The use of the term “at least one” will be understood to include one as well as any quantity more than one, including but not limited to, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, or more. The term “at least one” may extend up to 500 or 1000 or more, depending on the term to which it is attached; in addition, the quantities of 500/1000 are not to be considered limiting, as higher limits may also produce satisfactory results. In addition, the use of the term “at least one of X, Y, and Z” will be understood to include X alone, Y alone, and Z alone, as well as any combination of X, Y, and Z. The use of ordinal number terminology (i.e., “first”, “second”, “third”, “fourth”, etc.) is solely for the purpose of differentiating between two or more items and is not meant to imply any sequence or order or importance to one item over another or any order of addition.

The term “or combinations thereof” as used herein refers to all permutations and combinations of the listed items preceding the term. For example, “A, B, C, or combinations thereof” is intended to include at least one of: A, B, C, AB, AC, BC, or ABC; and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

The term “mobile device′ means a portable handheld device having a camera and a light source. For example, the mobile device and be a smartphone, tablet, or similar electronic device.

The term “nearly coaxial” refers to being coaxial or having approximately parallel axes with the axes being no more than about 4 mm of each other, or more typically no more than 3 mm, or 2.5 mm. By “approximately parallel” it is meant that the axes are parallel or at a slight angle to each other, wherein the slight angle is no more than 5°, more typically no more than 3°, or no more than 2°.

The term “pinhole” or “pinhole aperture” refers to a small circular hole having a diameter at least about 1 microns, more typically at least 3 microns, at least 5 microns or at least 10 microns, and no more than about 4 mm, more typically no more than 3 mm, or no more than 2.5 mm.

Turning now to the currently disclosed device and method, and the related figures, the disclosed device is an optical accessory for a mobile device such as a smartphone or tablet. The optical accessory may be comprised of optical elements, which can redirect light. For example, the optical elements can have a mirror surface and can be a mirror or can be a splitter or a prism. The device has a first optic element that redirects the mobile device's flashlight to a second optical element that redirects the light to a third optical element that redirects the light to be nearly coaxial with the smartphone camera in such a way to create an ophthalmoscope system which allows a view of the fundus oculi.

The first reflective surface redirects the flashlight so that the light is parallel to the smartphone. The second reflective surface redirects the light to move toward the mobile device camera. The third reflective surface redirects the light to move perpendicular to the phone and is nearly coaxial with the camera.

These light reflections are contained within an enclosed channel (also referred to as light channel) configured to prevent light scatter and promote light collimation within the channel. For example, the enclosed channel can have a width and height that are no more than about 5 mm, more typically no more than 4 mm, or no more than 3.5 mm, and typically will be at least 50 microns. While the channel can have any suitable cross-sectional shape (quadrilateral, oval, circular, etc.), The pinhole apertures associated with it will have a diameter that is equal to or less than the width and height (or diameter if applicable) of the channel, and more typically, the pinhole diameter will be smaller. The enclosed channel is oriented to run lengthwise parallel to the surface of the mobile device, thus, perpendicular to the viewing direction of the camera. For example, to help reduce light scatter the channel can have a dark color, such as black.

After the final re-direction of light with the third reflective surface, the light passes through a pinhole that is nearly coaxial with the camera's viewing direction to reduce divergent light and further promote light collimation.

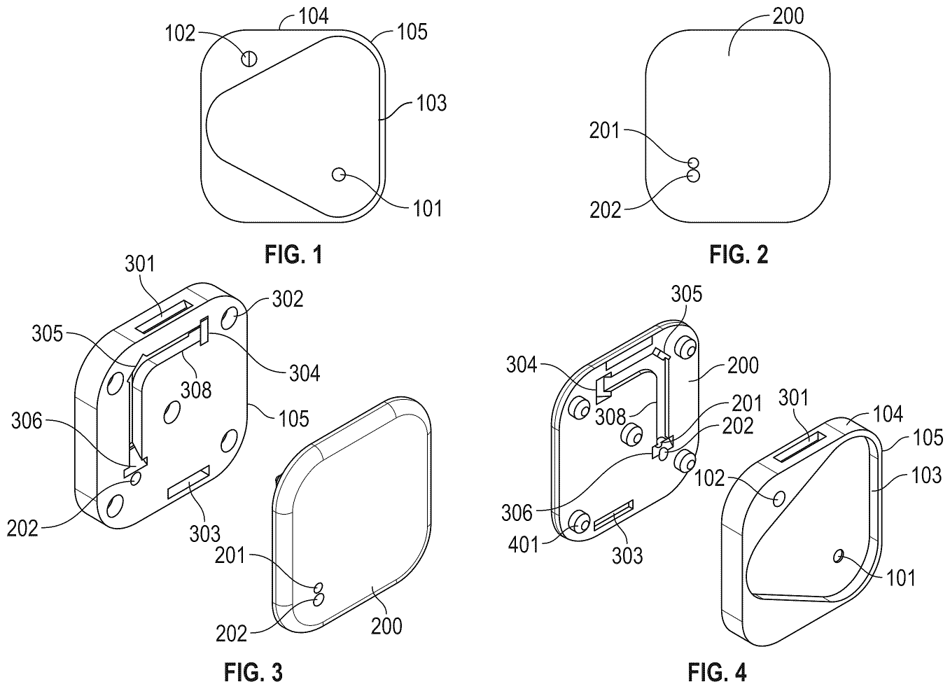

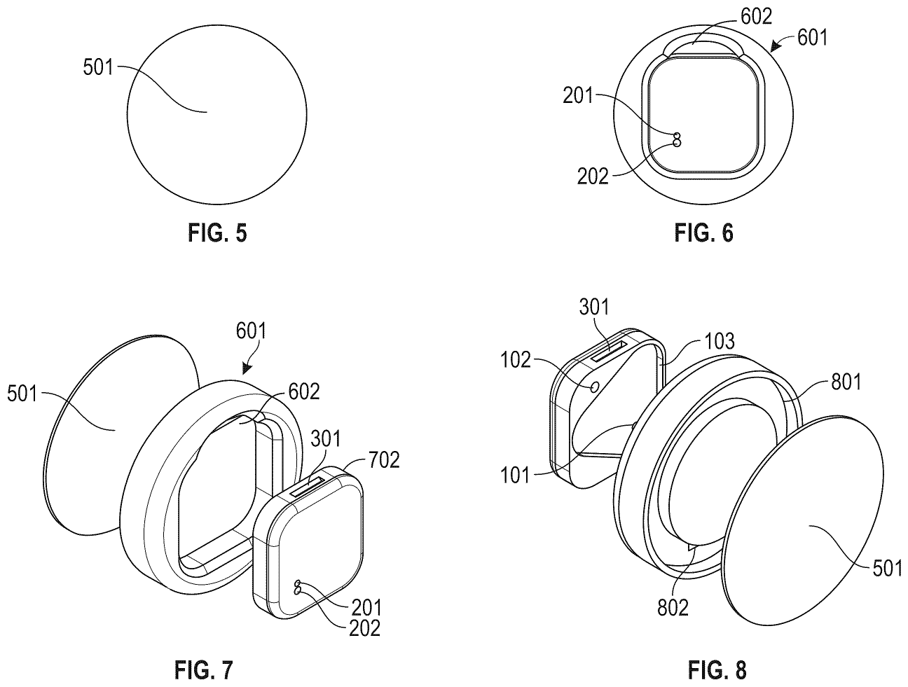

Turning now to figures, FIGS. 1 to 4 illustrate an ophthalmoscope optical accessory 100 according to this disclosure. The accessory 100 redirects the light from a light source 905 of a mobile device 900 to be in alignment with and nearly coaxial with the viewing direction of the mobile device camera 1002 to allow a view of the fundus oculi. FIGS. 5 to 8 illustrate a magnetic case 601 which is configured to allow the optical accessory 100 to magnetically, removably attach to the case 601. The case 601 then magnetically attaches to the native mobile device magnets (not shown).

Thus, as depicted in FIGS. 1 to 4, pinhole aperture 101 on a base plate 104 is configured to align with a mobile device camera 1002 on a surface 1004 of the mobile device 900 when the optical accessory 100 is attached to the mobile device 900. It has been discovered that pinhole 101 being flush or nearly flush with the mobile device camera 1002 provides for enhanced imaged clarity and sharpness. Accordingly, some embodiments will have a pinhole aperture 101, which is not greater than 0.4 mm from the camera lens surface, and more typically, no greater than 0.3 mm or no greater than 0.2 mm. A pinhole aperture 102 aligns with the light source 905 for the mobile device 900. The light source 905 is typically one usable as a flash and/or flashlight on the mobile device 900.

The rim 103 of the housing 105 of the optical accessory 100, typically on the base plate 104, allows for interfacing with the mobile device 900—its camera 1002 and light source 905—with a shape unique to each mobile device 900.

On the face plate 200 of the optical accessory 100, pinhole aperture 201 is provided for redirected light from the light source 905 of the mobile device 900 in alignment with and nearly coaxial to the cameral viewing direction, which in use will be toward subject's eye. An anterior pinhole aperture 202 for the camera 1002 of the mobile device 900 is also provided and in use faces toward the subject's eye.

The pinhole apertures 101, 102 on the base plate 104 will typically be the same size or greater than the pinhole apertures 201, 202 on the face plate 200. For example, light source pinhole aperture 102 could be about 3 mm with light source pinhole aperture 201 being 2.5 mm. While there will typically be a camera pinhole 101 and light source pinhole 102 on the base plate 104, the face plate 200 can have a single pinhole that serves as both camera pinhole 202 and light source pinhole 201, such as when the light from the light source 905 of the mobile device 900 is reflected to be coaxial with the camera 1002 viewing direction. Alternatively, the face plate 200 can have separate camera pinhole 202 and light-source pinhole 201, such as when the light from the light source 905 of the mobile device 900 is reflected to be nearly coaxial but not coaxial. Surprisingly, it has been found that the nearly coaxial (but not coaxial) arrangement results in better images than a coaxial alignment. It is believed that this is due to the nearly coaxial (but not coaxial) arrangement resulting in less glare than the coaxial arrangement.

As relating to two pinholes on the face plate 200, generally the coaxial distance will be greater than the combined radius of the first and second pinholes so that the pinholes do not overlap in some embodiment; however, in other embodiments the pinholes can have a slight overlap as long as they still are distinct; thus typically this will be an overlap of no more than 10% (or no more than 5%, or no more than 2%) of the smaller pinhole radius.

As best seen in FIGS. 3 and 4, the housing 105 of the optical accessory 100 can have a recessed finger grip 301 to allow for ease of removal of the optical accessory 100 from the case 601 and/or for ease of removal from the mobile device 900. Further, guidance holes 302 allow for interfacing with guidance hole studs 401 to ensure proper positioning and mating of the face plate 200 and the base plate 104.

The housing 105 has a recessed space 303 for receiving a magnet (not shown) used for attaching to the case 601 by interacting with a magnet 803 in magnetic channel 802 of the case 601.

First optical element 304, second optical element 305 and third optical element 306 are contained in the light channel 308, which forms into an enclosed channel with the face plate 200 and the base plate 104 are mated together.

The first optical element 304 redirects the light from the light source 905 of the mobile device 900 toward the second optical element 305. Subsequently, the second optical element 305 redirects the light received from first optical element 304 to the third optical element 306, the third optical element 306 receives the light from the second optical element 305 and redirects the light to be aligned with and nearly coaxial with the camera viewing direction, and hence when in use, towards the subject's eye.

Typically, the camera viewing direction will not be redirected but will pass straight through the housing 105. However, it will pass through at least one pinhole, and more typically through at least pinhole 101 and 202, and thereby image light from an object (the subject's eye or fundus oculi) undergoes collimation and a reduction in light scattering by passing through the pinhole(s) before reaching the camera 1002. It has been discovered that image sharpness and clarity is increased by using two pinholes 101 and 202 and having them relatively close to each other. For example, no greater than 10 mm, no greater than 8 mm, or no greater than 7 mm, but the two pinholes 101 and 202 will typically be spaced apart at least 3 mm, at least 4 mm, at least 5 mm, at least 6 mm. For example, the spacing can be about 6.25 mm. Generally, this spacing will also determine the thickness of the casing 702 so that the distance from the posterior surface (the surface interfacing with the camera) and the anterior surface (patient facing surface) is the same or about the same as the distance between pinhole 101 and 202.

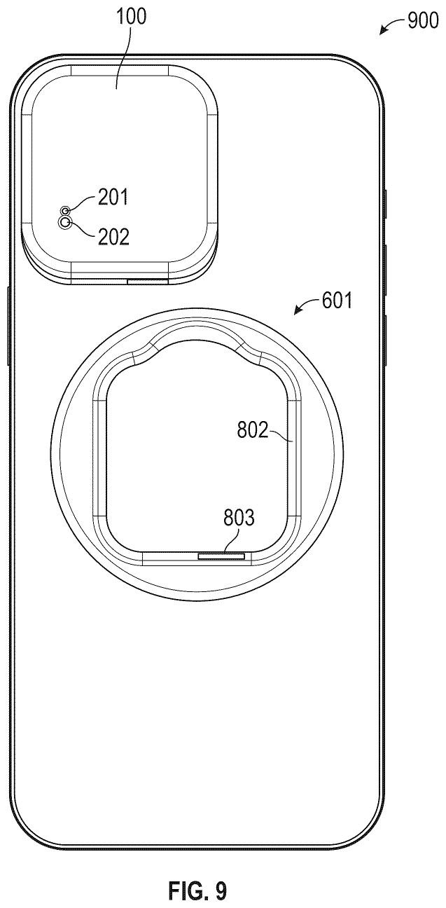

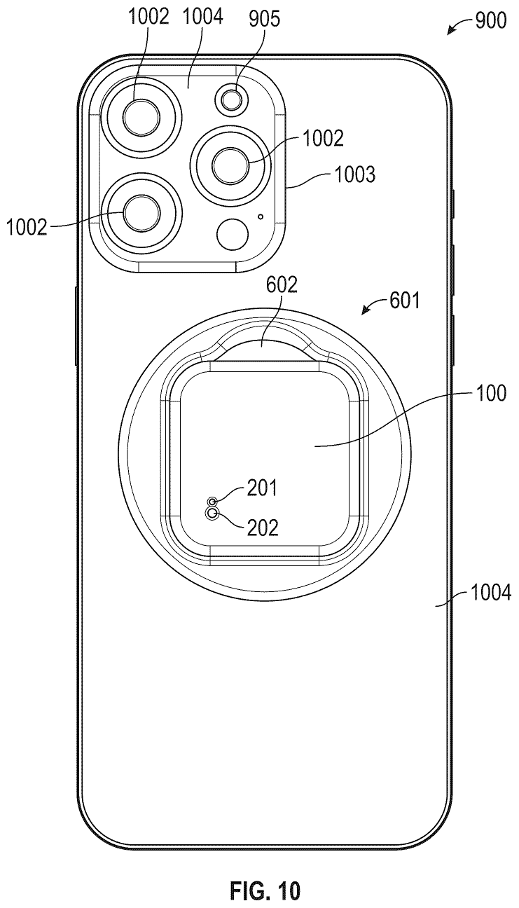

Turing now to FIGS. 5 to 11, magnetic case 601 is illustrated in relation to the optical accessory 100. The magnetic case 601 protects the optical accessory 100 when not in use. A rear protective cover 501 may be configured to cover the magnetic case 601 and optical accessory 100, and cover 501 may be positioned next to the mobile device surface 1004 when the case 601 is attached to the mobile device 900. Additionally, the case 601 can have a recessed section 602, which allows access to the recessed finger grip 301 of the optical accessory 100 to remove the optical accessory 100 from the magnetic case 601.

The case 601 has a channel 801 for magnetic element(s) (not shown) to be placed in the correct orientation for interfacing with native mobile device magnetic arrangement. Also, a channel 802 can be provided for magnetic element(s) to interface with one or more magnets 803 positioned within the recessed space 303 of the optical accessory 100.

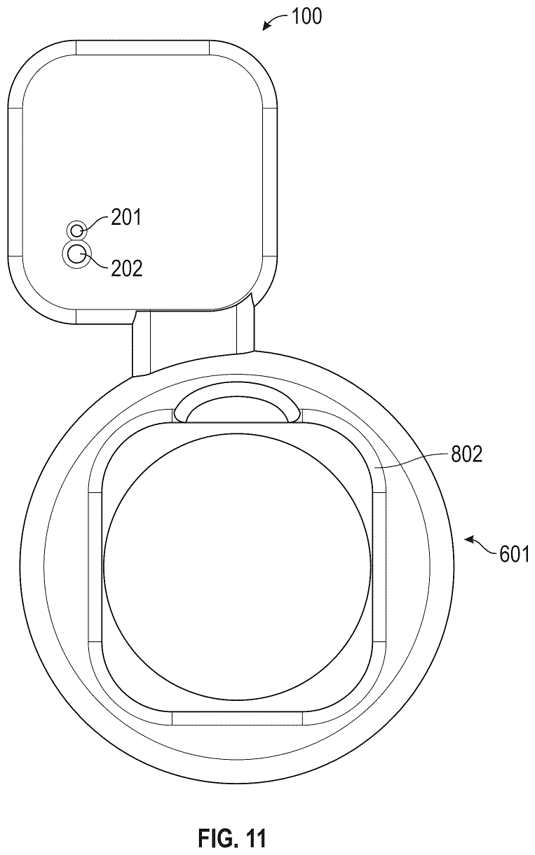

As can be seen from the figures, the case 601 attaches to the mobile device 900 separate from the location of the camera 1002 and light source 905 and holds the optical accessory 100 when not in use. When the optical accessory 100 is to be used, the optical accessory 100 is removed from the case 601 and positioned over the camera 1002 and light source 905. Typically, the optical accessory 100 can be pressure fitted over the camera 1002 and light source 905 by rim 103 of the housing 105 interfacing with the rim 1003 of the mobile device 900 about its camera 1002 and light source 905. In some embodiments, the case 601 has an exterior magnetic attachment which holds the optical accessory 100 in place in relation to the camera 1002 and light source 905, as shown in FIG. 11.

In operation, the light from the light source 905 of the mobile device 900 enters the optical accessory 100 through the pinhole aperture 102. This light interfaces with first optical element 304 to redirect the light parallel to the surface of the mobile device 900 (perpendicular to the viewing direction of the camera) through the light channel 308 towards second optical element 305. The light is then redirected from second optical element 305 through the light channel 308 towards third optical element 306. The third optical element 306 redirects the light towards the subject's eye through the pinhole 201. This light has now been redirected to be aligned with a nearly coaxial with the viewing direction of the mobile device camera 1002.

The mobile device camera 1002 is able to view the subject's eye through the optical accessory's base plate pinhole 101 and the optical accessory's face plate pinhole 202. The close proximity of the redirected light in pinhole 201 to the mobile device camera pinhole 202 allows for the light to be in line with the camera 1002 to allow a view of the fundus oculi.

In embodiments, the first, second, and third optical elements 304, 305, 306 are mirrors. These mirrors are oriented at 45 degrees to allow for the light to be redirected 90 degrees. The light from the mobile device light source 905 enters the optical accessory's pinhole 102 and is redirected 90 degrees by the first mirror 304 to travel to the second mirror 305, which redirects the light 90 degrees to the third mirror 306 which redirects the light another 90 degrees through pinhole 201 towards the subject's eye.

In embodiments, the optical accessory incorporates a magnet(s) within recess 303 which allows for attachment to the case magnet(s) 803 positioned within the magnetic channel 802. The case has another magnet(s) in channel 801, which allows the case to attach to the mobile device's native magnets.

In accordance with the above, the current optical accessory provides for increase collimation of the light from the light source and the image light to the camera, and reduces interference called by light scattering. This is at least partially accomplished by the use of multiple pinholes as described herein. These pinholes advantageously collimate the light and allow for un-dilated exams of the fundus oculi.

Other embodiments of the present invention will be apparent to one skilled in the art. As such, the foregoing description merely enables and describes the general uses and methods of the present invention. Accordingly, the following claims define the true scope of the present invention.

What is claimed is:

1. An optical accessory for a mobile device having a device surface containing a light source and a camera having a viewing direction, the optical accessory comprising:
a light channel containing a first optical element, a second optical element and a third optical element, wherein the first optical element is configured to reflect light from the light source to the second optical element, wherein the second optical element is configured to reflect light to the third optical element, wherein the third optical element is configured to reflect light toward a fundus oculi of an eye to be examined,
wherein the first optical element and second optical element direct light from the light source along the light channel and parallel to the surface, and the third light element reflects light from the light channel in a direction perpendicular to the device surface such that light from the third light element is in alignment with and nearly coaxial with the viewing direction of the camera in such a way as to direct the light to the fundus oculi of an eye to be examined by the camera to thus create an ophthalmoscope system which allows a view of the fundus oculi.

2. The optical accessory according to claim 1, further comprises a housing having a face plate and a base plate, wherein the face plate and the base plate together form the light channel and wherein the base plate is configured to be adjacent to the device surface when the optical accessory is attached to the mobile device.

3. The optical accessory according to claim 2, wherein the face plate defines a first pinhole aperture aligned with the third optical element such that light reflected from the third optical element passes through the first pinhole and is collimated by passing therethrough.

4. The optical accessory according to claim 2, wherein the face plate defines a second pinhole aperture aligned with the camera when the optical accessory is attached to the mobile device such that image light from the eye passes through the second pinhole aperture prior to entering the camera and is collimated by passing through the second pinhole aperture.

5. The optical accessory according to claim 3, wherein the face plate defines a second pinhole aperture aligned with the camera when the optical accessory is attached to the mobile device such that image light from the eye passes through the second pinhole aperture prior to entering the camera and is collimated by passing through the second pinhole aperture.

6. The optical accessory according to claim 2, wherein the base plate defines a third pinhole aperture aligned with the light source when the optical accessory is attached to the mobile device such that light from the light source passes through the third pinhole to the first optical element and is collimated by passing therethrough.

7. The optical accessory according to claim 2, wherein the base plate defines a fourth pinhole aperture aligned with the camera when the optical accessory is attached to the mobile device such that the image light passes through the fourth pinhole aperture prior to entering the camera and is collimated by passing through the second pinhole aperture.

8. The optical accessor of claim 3, wherein the first pinhole aperture, second pinhole aperture, third pinhole aperture and/or fourth pinhole aperture are configured to reduce light scatter.

9. The optical accessory according to claim 1, wherein the first optical element, the second optical element and third optical element are a mirror, beam splitter or prism.

10. The optical accessory according to claim 1, wherein the light channel is configured to reduce light scatter and make the light more collimated.

11. The optical accessory according to claim 1, further comprising one or more magnetic attachments so that the optical accessory magnetically attaches to the mobile device.

12. A method for obtaining images of a fundus oculi of an eye to be examined, the method comprising:
attaching an optical accessory to a mobile device having a device surface containing a light source and a camera having a viewing direction, wherein the optical accessory comprises a light channel containing a first optical element, a second optical element and a third optical element; and wherein the optical accessory is aligned with the mobile device so that the first optical element is aligned with the light source so as to receive light from the light source;
introducing light from the light source to the first optical element,
reflecting light received by the first optical element to the second optical element through a first portion of a light channel that is parallel with the device surface;
reflecting light from the second optical element to the third optical element through a second portion of the light channel that is parallel with the device surface;
reflecting light from the third optical element toward the fundus oculi so as to produce image light of the fundus oculi, wherein the third light element reflects light from the light channel in a direction perpendicular to the device surface such that light from the third light element is in alignment with and nearly coaxial with the viewing direction of the camera;
receiving image light from the fundus oculi in such a way to create an ophthalmoscope system which allows a view of the fundus oculi so as to produce an image with the camera.

13. The method according to claim 12, wherein the optical accessory further comprises a housing defining one or more pinholes and wherein the method further comprising passing the light from the light sources through at least a first pinhole so as to collimate the light and reduce light scatter.

14. The method according to claim 13, wherein the method comprises passing the image light through at least a second pinhole so as to collimate the image light and reduce light scatter.

15. The method according to claim 12, wherein the light channel is configured to reduce light scatter and make the light more collimated.

16. The method according to claim 12, wherein the optical accessory includes one or more magnetic attachments and the step of attaching the optical accessory to the mobile device is by magnetically attaching.
